SEQUENCE LISTING (8 sequences)

SEQ ID NO: 1 - DNA, 540 bp, Plasmodium falciparum
atggcgtgccaagttgataacccccctaaaacatacccaaacgataaaacagctgaatacgaaaagtacgcaaattatatgaactatctatattattatcaaaataatgaattaaaaaaaatcgattcctcttattttaaagataaatatttaggattattttttggagcttcatggtgtaaatactgtgtaacctttatagatagcttaaatatatttaaaaagaacttccccaatgttgaaattatatatataccatttgatagaacatatcaagagtaccaatcctttttaaaaaatacaaacttttatgctttaccttttgataattatttatatatatgtaaaaagtatcaaataaaaaatctaccttcctttatgttaattacacctaataataatatactagtaaaggatgcagcacaattaattaaaacagatgaatatataaataatttaaaatcattaataaaaaattatatcatacatcctaaaacgtttcaatttaataatcgcttttttgatttgtttcgtaattga

SEQ ID NO: 2 - protein, 179 aa, Plasmodium falciparum
MACQVDNPPKTYPNDKTAEYEKYANYMNYLYYYQNNELKKIDSSYFKDKYLGLFFGASWCKYCVTFIDSLNIFKKNFPNVEIIYIPFDRTYQEYQSFLKNTNFYALPFDNYLYICKKYQIKNLPSFMLITPNNNILVKDAAQLIKTDEYINNLKSLIKNYIIHPKTFQFNNRFFDLFRN

SEQ ID NO: 3 - DNA, 3946 bp, Plasmodium falciparum
atggcaccgaaaaatggaagtagaaatggaaaattacttagtttaagggatgttctggaaaatattggaagcggcataaaagataagagaaaaaatcagagtaaatatacagataaattgaaagggatattaacaaaagcaaaatttgttgatggattgagtagtagatatggttatgtaagggattctgatggaatttcatgtaatcttagtcacaaattccatactaatataacaattgaagctgcaagggatccttgttatggaagggaacaaaaccgttttgatgaaaatgtcgaatcgtattgtaacaatgataaaataagaggtagtgggaaaatatttgatggaagagtatgtgtcccacctagaaggcaacatatatgtgatcataatttagaatatttaaataacaataactgatgacactgatgatttgttgggaaatgtgttagttacagcaaaatatgaaggtcaatctattgttaataatcatccacataaagaaacttctgatgtttgtactgctcttgcacgaagttttgctgatataggtgacattgtaagaggaatagatatgtttaaacctaatgaccaagacgaagtatggaatggtctaaggtcagttttcaagaaaatacatgataatttgtcatctgaagtaaaaaatgcttatccagatgatggatctggaaattattttaaattaagggaagattggtggacagcgaacagagatcaagtatggaaagccatgacttgtgttgcaccagaaaatgcttattttagaaaaacagaagctgatggaataggaatttcaagtttaattttaccatattctaaatgtggacgtgatactgacccccctgttgttgattatatccctcaacgcttaagatggatgagtgaatggtctgaatatttctgtaatgtattaaataaagaaatagatgaaatgaataatcaatgtaaagattgtgaaatgagccgaagatgcaatgatgatagcgaagggggaaaatgtaaaaaatgcaaagaacaatgtcaaatattcaaggagctcgtaagtaaatggaaaaaccaatttgataaacaatcaatgaaatatatggaattatataataaagcaagtactaatataactaaacagaactctagtgcacctgaacgtggatatcgacgtaatcatagacgtagaggttacgatgatgatacaaatgtacaattatttttgaaaaaagtaatagaaaataatgagtgtaaagttgagtcccttggaaaatatcttgataaaacaagtcattgtggtaattataattttaattatgataatactccaggttccaatagatctaacgcttttgaaataactccagaaaagtttaaaaaggcttgcaaatgtaaaatacctaatccattagaaaaatgtcctaatgaagaaaacaaaaatgtatgcacaagattcgataaggtttattcatgtacatcactttcttttaaaaatgacttgagcgaatggaataattcaggagtaaaaaataaagaaaatgacaataatggtgtgttagttcctcctagaagacgaaatttatgcataaatttgttttcaaaaaaagattataaaatgaaagatgaaaacgatttcaaagaggatctacttaatgctgcttttagtcaaggaaaattgttaggaaagaaatatagtaactacagtaatgaagcatatgaggctatgaagttcagttatgctgattacagtgatatcgtgaaaggtaccgacatgatgaatgatttaaaaaaattaaataaagaactaaatacacttcttaaagaaactgaaaaaggagatatatctgtggatcgtaaaacatggtgggatgataataaaaatgttgtatggaatgctatgttatgtggctataaaaccgaaaatgaaaatcaacaattgaattcatcgtggtgtaatgtacctgatgatgattatattgatcaatttttgagatggttaactgaatgggcccaacaatattgtaaagaaaaattaattaaagcacatataataaatacaaaatgtaaagatatcgttgaagggagaaaacataaaagtatggttgatataacagatgtagaatgtaaacgattatttattgattatgaagaatggtttcgttaccgatataatcaatggaagggattatctgaaaaatacattaagattaagaagagcaaaaattctggagtgaatataccctctgaggaatgtgctgcatcatacgtaacaaaacattgcaatggatgtatttgtaatttgagagatatggaggatatacataaaaacattaataaccaaaatgaattaatgaaggaaatgattaatataattaaatttgatactgatcaatatagaactcaattacaaaatatatcaaattctatggaaataaatccaaaaagtgtaaaaacagcagtagatactacgaaagatatagtttcatatggattggccggtactatgggagttgcagcaattggattacaagcaggagattttcttggaaaaaaaattcaagatttgtacaatgaatttatgaaacctgttgaaaaaaaattagatacatcatctaaaaatcttaatatctacgaagaccccaacattatggttcctgctggtattggtgtcgccttaactctaggattgttattatttaagatgagaagaaaagcaaaacgtcaagtagatatgatacggatattacaaatgtcacaaaacgaatatggaattccgacaaccaaatcaccaaacaaatatgttccatatgggagtcaacgatataaaggcaaaacatacttatatgttgaaggagatacagacgaagagaaatatatgtttatgtctgatactactgatataacctcttccgaaagtgaatatgaagaaatggatatcaatgatatatatgttcctggtagtccaaaatacaaaacgttgatagaagttgttctggagccatcaaaaagagatacacaaaatgatatacctagtgataatacacctagttataaacttacagatgaggaatggaatcaattgaaagatgattttatatcacaatatttaccaaatacagaaccaaataataattatagaagtggaaatagtccaacaaataccaataatactaccacgtcacatgataatatgggagaaaaaccttttattatgtccattcatgatagaaatttatatactggagaagaaattagttataatattaatatgagtactaatactatggatgatccaaaatatgtatcaaataatgtatattctggtattgacctaattaatgattcattaaatagtggtaatcaacctattgatatatatgatgaagtgctaaaaagaaaagaaaatgaattatttggaacaaatcatgtgaaacaaacgagtatacatagtgttgcaaaaaatacatatagtgacgacgctataacaaataaaataaatttgttccataaatggttagatagacatagagatatgtgtgaaaagtgggaaaatcatcatgaacgtttagctaaattaaaagaaaaatgggaaaatgataatgatggaggtaatgtacctagtggtaatcatgtgttgaatacggatgtttcgatcgaaatagatatggataatcctaaacctataaatcaatttagtaatatggatataaacgtggatacacctactatggataatatggaagatgatatatattatgatgtaaatgataatgatgatgataatgatcaaccatctgtgtatgatatacctatggatcataataaagtagatgtagatgtacctaagaaagtacatattgaaatgaaaatccttaataatacatctaatggatcgttggaacaacaatttcctatatcggatgtatggaatatataa

SEQ ID NO: 4 - protein, 1315 aa, Plasmodium falciparum
MAPKNGSRNGKLLSLRDVLENIGSGIKDKRKNQSKYTDKLKGILTKAKFVDGLSSRYGYVRDSDGISCNLSHKFHTNITIEAARDPCYGREQNRFDENVESYCNNDKIRGSGKIFDGRVCVPPRRQHICDHNLEYLNNNNTDDTDDLLGNVLVTAKYEGQSIVNNHPHKETSDVCTALARSFADIGDIVRGIDMFKPNDQDEVWNGLRSVFKKIHDNLSSEVKNAYPDDGSGNYFKLREDWWTANRDQVWKAMTCVAPENAYFRKTEADGIGISSLILPYSKCGRDTDPPVVDYIPQRLRWMSEWSEYFCNVLNKEIDEMNNQCKDCEMSRRCNDDSEGGKCKKCKEQCQIFKELVSKWKNQFDKQSMKYMELYNKASTNITKQNSSAPERGYRRNHRRRGYDDDTNVQLFLKKVIENNECKVESLGKYLDKTSHCGNYNFNYDNTPGSNRSNAFEITPEKFKKACKCKIPNPLEKCPNEENKNVCTRFDKVYSCTSLSFKNDLSEWNNSGVKNKENDNNGVLVPPRRRNLCINLFSKKDYKMKDENDFKEDLLNAAFSQGKLLGKKYSNYSNEAYEAMKFSYADYSDIVKGTDMMNDLKKLNKELNTLLKETEKGDISVDRKTWWDDNKNVVWNAMLCGYKTENENQQLNSSWCNVPDDDYIDQFLRWLTEWAQQYCKEKLIKAHIINTKCKDIVEGRKHKSMVDITDVECKRLFIDYEEWFRYRYNQWKGLSEKYIKIKKSKNSGVNIPSEECAASYVTKHCNGCICNLRDMEDIHKNINNQNELMKEMINIIKFDTDQYRTQLQNISNSMEINPKSVKTAVDTTKDIVSYGLAGTMGVAAIGLQAGDFLGKKIQDLYNEFMKPVEKKLDTSSKNLNIYEDPNIMVPAGIGVALTLGLLLFKMRRKAKRQVDMIRILQMSQNEYGIPTTKSPNKYVPYGSQRYKGKTYLYVEGDTDEEKYMFMSDTTDITSSESEYEEMDINDIYVPGSPKYKTLIEVVLEPSKRDTQNDIPSDNTPSYKLTDEEWNQLKDDFISQYLPNTEPNNNYRSGNSPTNTNNTTTSHDNMGEKPFIMSIHDRNLYTGEEISYNINMSTNTMDDPKYVSNNVYSGIDLINDSLNSGNQPIDIYDEVLKRKENELFGTNHVKQTSIHSVAKNTYSDDAITNKINLFHKWLDRHRDMCEKWENHHERLAKLKEKWENDNDGGNVPSGNHVLNTDVSIEIDMDNPKPINQFSNMDINVDTPTMDNMEDDIYYDVNDNDDDNDQPSVYDIPMDHNKVDVDVPKKVHIEMKILNNTSNGSLEQQFPISDVWNI

SEQ ID NO: 5 - DNA, 1242 bp, Plasmodium falciparum
atgaaagtcggaaaattaaaaaagagaaaaaattctggcttgttatatccatattttaagaataaatcatttagattaaatagatatatattcataaaaccaataaaaagtgtaaaactaaattataagaaaaaaaaaatgaacctaacacatgaaatttgtattttaaattgtagtgaaaaattaatagattataaactagcttttcaacttcaaaatatcctacatcattcaaaaattattatgaaaaataaaaatgaagtacaaatatcaaatcatttagaattaaaaaaattcaaaaatttcaaagaaaacatggaaaaatatgatttctgttttatattacaacatactccatgctataccttaggtagtgtagcaaattgtagtgatatacttctagataaggaaaattattatattgaagaattaggagatatatataataatttgtattcgaatgaaattattcatcttatgaataaatgtgaaacaattcaagataaaattaatcaatctgatatatataatgaaaatacaaattatttcaataattttttaaaacattgtagacaaagaaaaatacccatttatcgagttaacaggggaggcaaagctacataccatggacctggacagttagtattatattttatatttaacttaaaaaattatccatccaattataatgagcgaattataaataagcactataaatatacaaacaaagaaaactttccatcaaaaacatcggaatatgaaaaaaataacatatatacaaattcaaacagtaaagaaaacatatcatctatagaacgcacttttgatttgcgcacaacaataaataactttcaaaaaattggaatggaaaccttgcaaaaatttaatataaaaacacactgtaaaaaagatacaataggtatcttttataaggataaaaaaattatatccataggattgaaaataacaaaatatatatctatgcatggattgtcattaaattttaatctcgataacaattttttaaaatatctattatcatgtggtatgaatcataatgattatatatccatgcatgaaataaatgaaatgaaaaaaaaaaattatatttatcaaaaaggaaaaatagctagtagctcaaatatattaaatgaattaactttaaatataacagagtcattaaaaaaggtgtttaatgtaaaagtaagaaatataaaagatatacgagaaatgttttattaa

SEQ ID NO: 6 - protein, 413 aa, Plasmodium falciparum
MKVGKLKKRKNSGLLYPYFKNKSFRLNRYIFIKPIKSVKLNYKKKKMNLTHEICILNCSEKLIDYKLAFQLQNILHHSKIIMKNKNEVQISNHLELKKFKNFKENMEKYDFCFILQHTPCYTLGSVANCSDILLDKENYYIEELGDIYNNLYSNEIIHLMNKCETIQDKINQSDIYNENTNYFNNFLKHCRQRKIPIYRVNRGGKATYHGPGQLVLYFIFNLKNYPSNYNERIINKHYKYTNKENFPSKTSEYEKNNIYTNSNSKENISSIERTFDLRTTINNFQKIGMETLQKFNIKTHCKKDTIGIFYKDKKIISIGLKITKYISMHGLSLNFNLDNNFLKYLLSCGMNHNDYISMHEINEMKKKNYIYQKGKIASSSNILNELTLNITESLKKVFNVKVRNIKDIREMFY

SEQ ID NO: 7 - DNA, 1704 bp, Plasmodium falciparum
atgagaaggtacctgttgattacctgtttgtttgtcctgtgttgcttaaaattaaagcatgtgaactttttaaagtgggagcaggaaaatgatttttattatataaataatgagaaactattaaaaagggtattacataatgtagaacaaactaaagaaagaacagaagttgataaaccaatagtatttggtataaggaaaggaaaatttgttacaatacacaaagaaacaaaagaagagaagatgctgaaggataatttgatagaagctatattatttgatcctaagaaagatgaagaattaaaaattgatataaaagaaacaaatatagataaagatagtaaaaaaaaacaaaaaagagaaaatggaattattaaagatgatacagctaaggataaggatttgtattcatatactaaagacccgattactctccataaaaaaaaattaaaagaagaaaagaattttgttatgatcaaagaatttgtaaaagatttatctagtcgagatgaaaatgtattaatatctaatgtgaacatttttttaaaaagaatatttaatttgatattgagggaaaaaataattactgcaatgtgttcagatgtacaaaatgaaggaatagaaaataataacacacaaatgaagggcaaacaaataaaggacgcacaaatgaagggcaaacaaaataataacacacaaatgaagggcaaacaaaataataacacacaaatgaagggcaaacaaaataataacacacaaatgaatgacgcacaaatgaatgacgcacaaaattatgatggcaaagataacaattcagaatgcttgaaaaataataagaattgtaatttcgataacaaaatcaagattaaagattgtagtaagggttccataagttgttttctctcgaacattaaaaatgaagaattttataaagctccagatttatttaaatattatatatctttagaaaaaatgttgaggagctcttctgttcgatccaaaacagacaggatatcaaaatattttactttttatccagtatctatggataaagaatattatgaagagaaaataaataatcatgtatttttagaggctgttagaaatatattatttgatttagatgaaggaaataaaaaggataaaaaaaaggttttttcgagttttgtaatagtcgtagatacattaatatctttaataaaaaaagaaaaggtagtaaaagaaatgtatatgtttatacatttattttttcaagatttaaatttattaaataaaaaaatattagacattttattaaaaagttcttttaagccaggagcatcatttaatattccagatttcaataagaaaaatttcgaatttattttatcaagaatatatacaagatatgttttaaataatttattaaataagacattcaataattcagataccatcaatatgtctgattttttaaataacaaaataaaacctttcaattttagttttacggaaacaagtgtaaacttgctaaagaatgagggtattcagataaaggatgatgaccttttggtgagcgaagaaaatttgtgtaaatatatacctatcaaaaaaaaattattatatgaaaaacttaacaagacaaggaaagctgcagaggaagctatactggattatatatttagacttttattaagaaaattacatgaatttataacagaataa

SEQ ID NO: 8 - protein, 567 aa, Plasmodium falciparum
MRRYLLITCLFVLCCLKLKHVNFLKWEQENDFYYINNEKLLKRVLHNVEQTKERTEVDKPIVFGIRKGKFVTIHKETKEEKMLKDNLIEAILFDPKKDEELKIDIKETNIDKDSKKKQKRENGIIKDDTAKDKDLYSYTKDPITLHKKKLKEEKNFVMIKEFVKDLSSRDENVLISNVNIFLKRIFNLILREKIITAMCSDVQNEGIENNNTQMKGKQIKDAQMKGKQNNNTQMKGKQNNNTQMKGKQNNNTQMNDAQMNDAQNYDGKDNNSECLKNNKNCNFDNKIKIKDCSKGSISCFLSNIKNEEFYKAPDLFKYYISLEKMLRSSSVRSKTDRISKYFTFYPVSMDKEYYEEKINNHVFLEAVRNILFDLDEGNKKDKKKVFSSFVIVVDTLISLIKKEKVVKEMYMFIHLFFQDLNLLNKKILDILLKSSFKPGASFNIPDFNKKNFEFILSRIYTRYVLNNLLNKTFNNSDTINMSDFLNNKIKPFNFSFTETSVNLLKNEGIQIKDDDLLVSEENLCKYIPIKKKLLYEKLNKTRKAAEEAILDYIFRLLLRKLHEFITE

(19) United States
(12) Patent Application Publication
(10) Pub. No.: US 2012/0328645 A1
(43) Pub. Date: Dec. 27, 2012

(54) Malaria Vaccines

(76) Inventors: BENJAMIN U. HOFFMAN, ROCKVILLE, MD (US); ANUSHA GUNASEKERA, ROCKVILLE, MD (US)

(21) Appl. No.: 12/945,450

(22) Filed: Nov. 12, 2010

Related U.S. Application Data
(63) Continuation of application No. 12/405,066, filed on Mar. 16, 2009, now abandoned.
(60) Provisional application No. 61/069,394, filed on Mar. 14, 2008.
(60) Provisional application No. 61/127,593, filed on May 13, 2008.

(51) Int. Cl. A61K 39/015; A61P 33/06

(52) U.S. Cl. 424/191.1

(57) ABSTRACT

Transcript profiles for irradiated and non-irradiated P. falciparum sporozoites were compared in an attempt to identify transcripts that are reproducibly perturbed by irradiation. Four loci were identified with high confidence. Three of these transcripts were derived from 3 different known gene families, and the fourth from a gene encoding a conserved hypothetical protein of unknown function. All four loci are up-regulated in radiation attenuated sporozoites which have been shown to be very effective in establishing protective immunity against malaria. Up-regulation of these transcripts contributes directly to protective immunity. The polypeptides encoded by the transcripts, individually and/or in combination, are incorporated into subunit vaccines, useful for the prevention of malaria and the establishment of protective immunity against malaria.

### CROSS REFERENCE TO RELATED APPLICATIONS

This application claims priority from U.S. Nonprovisional application Ser. No. 12/405,066, filed Mar. 16, 2009. U.S. Nonprovisional application 12/405,066, filed Mar. 16, 2009 claims priority to U.S. Provisional application 61/069,394, filed Mar. 14, 2008 and from U.S. Provisional application 61/127,593, filed May 13, 2008.

### REFERENCE TO A SEQUENCE LISTING SUBMITTED ELECTRONICALLY VIA EFS-WEB

The content of the electronically submitted sequence listing (Name: sequence listingsascii.txt, Size: 31,696 bytes; and Date of Creation: Nov. 12, 2010) filed herewith with the application is incorporated by reference in its entirety

### BACKGROUND OF THE INVENTION

1. Field of the Invention

This invention relates to genes which are expressed at the sporozoite stage of the Plasmodium lifecycle. More specifically, the invention relates to Plasmodium genes, the expression of which is differentially regulated in radiation attenuated sporozoites, and the use of these genes and their gene products in the prevention of malaria.

2. Background Art

Plasmodium-caused malaria kills more children (>1 million) annually, more than any other single infectious agent (1). Experimentally-induced sterilizing protection in humans of >90% against malaria has, to date, been conferred only via immunization with metabolically active, radiation-attenuated Plasmodium sporozoites. Irradiation of sporozoites prevents their completion of the hepatocyte stage cycle. However, the mechanism behind this attenuation, as well as the effects of irradiation on sporozoite gene expression and immunogenicity are not known.

Immunization of mice with radiation attenuated Plasmodium yoelli sporozoites confers greater than 90% protective immunity in these mice (2). Immunization of humans by the bite of mosquitoes carrying radiation attenuated Plasmodium falciparum sporozoites confers greater than 90% protective immunity against malaria (3). A major target of the protective immunity induced by irradiated sporozoites is the circumsporozoite protein, the major protein on the surface of sporozoites. In fact the most advanced malaria vaccine, RTS,S (5) only includes one P. falciparum protein, the PfCSP. However, it is now known that mice that cannot mount an immune response against the P. yoelii CSP can be completely protected against malaria by immunization with radiation attenuated P. yoelii sporozoites (4,6). Thus, there must be other proteins expressed in radiation attenuated P. yoelii sporozoites that are the targets of the protective immunity. This has been presumed for many years, but only recently definitively proven (4,6). Researches have undertaken efforts to identify other targets of protective immunity expressed by radiation attenuated sporozoites. One example of an identified target is P. falciparum sporozoite surface protein 2 (PfSSP2), also known as Thrombospondin adhesive protein (TRAP). The rationale and initial work establishing the importance of this protein and the path toward development of it as a vaccine was established more than 15 years ago (13). Another protein identified more recently is PfSPATR with a similar path toward development as a vaccine was established (14). PfSSP2 is being developed by multiple groups as a vaccine and subunit recombinant vaccines. Recombinant protein, recombinant virus (15), DNA vaccine (16), and prime boost (17) approaches have all been assessed in humans. None of the previously-identified protein targets under development, as described above, has ever been shown to have increased expression in radiation attenuated P. falciparum sporozoites as compared to non-irradiated sporozoites.

In an attempt to understand the mechanism behind attenuation, gene expression in sporozoites has been studied. The effect of irradiation (150 Gy) on 10 known and well-described genes was examined by qualitative reverse transcription polymerase chain reaction (RT-PCR) (8). Quantitative RT-PCR was used to assess expression of the same 10 genes (9). Although a few genes were up-regulated or down-regulated, most genes did not appear to be significantly affected at the transcription level. These studies provided the foundation for the current work, which characterizes regulation of gene expression in response to irradiation on a global scale. Transcript profiles were compared between irradiated and non-irradiated P. falciparum sporozoites to identify genes that are reproducibly up-regulated or down-regulated by irradiation (150 Gy). Four such genes are provided herein. Their gene products are pivotal in the protective immunity of radiation-attenuated sporozoites and provide compelling arguments as vaccine candidates.

### BRIEF SUMMARY OF THE INVENTION

Transcript profiles for irradiated and non-irradiated P. falciparum sporozoites were compared in an attempt to identify transcripts that are reproducibly differentially expressed by irradiation (150 Gy). Four loci were identified with high confidence. Three of these transcripts were derived from 3 different known gene families, and the fourth from a gene encoding a conserved hypothetical protein of unknown function. All four loci are up-regulated in radiation attenuated sporozoites which have been shown to be very effective in establishing protective immunity against malaria.

Up-regulation of the gene products of these transcripts contributes directly to protective immunity. The polypeptides encoded by the transcripts, individually and/or in combination, are incorporated into subunit vaccines, useful for the prevention of malaria and the establishment of protective immunity against malaria.

### BRIEF DESCRIPTION OF THE DRAWINGS/FIGURES

FIG. 1—Steps in the fabrication of microarrays.

FIG. 2—Production of irradiated and non-irradiated sporozoites and sporozoite RNA.

FIG. 3—cDNA synthesis, labeling and hybridization.

FIG. 4—Correlation of Cy5/Cy3 ratios between technical replicate arrays: Normalized Cy5/Cy3 ratios from each technical replicate within an experimental batch were log transformed and compared among each other. Shown here are ratios from Array 1 plotted against Array 2 (Batch 1) and the correlation coefficient squared (r2).

FIG. 5—Comparison of differentially regulated genes between technical replicates: Venn diagram depicting the number of shared genes that were up-regulated in response to radiation in all three technical replicate arrays derived from batch B1* (arrays 9,10,11). Each array is depicted as a circle with the number of genes specified within.

FIG. 6—Comparison of up-regulated genes between experimental batches:

Venn diagram depicting the number of genes that were consistently up-regulated in response to radiation between all three experimental batches (B1, B1* and B3). The shared list of genes within an experimental batch is depicted as a circle with the number of genes specified within.

SEQUENCE IDENTIFICATION
I - Olgo ID: C115-PlasmoDBID: PFC0166w
SEQ ID NO: 1
Predicted DNA/mRNA Sequence Sequence Length: 540 bp
ATGGCGTGCCAAGTTGATAACCCCCCTAAAACATACCCAAACGATAAAACAGCTGAATAC
GAAAAGTACGCAAATTATATGAACTATCTATATTATTATCAAAATAATGAATTAAAAAAA
ATCGATTCCTCTTATTTTAAAGATAAATATTTAGGATTATTTTTTGGAGCTTCATGGTGT
AAATACTGTGTAACCTTTATAGATAGCTTAAATATATTTAAAAAGAACTTCCCCAATGTT
GAAATTATATATATACCATTTGATAGAACATATCAAGAGTACCAATCCTTTTTAAAAAAT
ACAAACTTTTATGCTTTACCTTTTGATAATTATTTATATATATGTAAAAAGTATCAAATA
AAAAATCTACCTTCCTTTATGTTAATTACACCTAATAATAATATACTAGTAAAGGATGCA
GCACAATTAATTAAAACAGATGAATATATAAATAATTTAAAATCATTAATAAAAAATTAT
ATCATACATCCTAAAACGTTTCAATTTAATAATCGCTTTTTTGATTTGTTTCGTAATTGA
SEQ ID NO: 2
Predicted Protein Sequence
Sequence Length: 179 aa
MACQVDNPPKTYPNDKTAEYEKYANYMNYLYYYQNNELKKIDSSYFKDKYLGLFFGASWC
KYCVTFIDSLNIFKKNFPNVEIIYIPFDRTYQEYQSFLKNTNFYALPFDNYLYICKKYQI
KNLPSFMLITPNNNILVKDAAQLIKTDEYINNLKSLIKNYIIHPKTFQFNNRFFDLFRN
II - Oligo ID: I14393_1-PlasmoDB ID: PFI1820w
SEQ ID NO: 3
Predicted DNA/mRNA Sequence Sequence Length: 3948 bp
ATGGCACCGAAAAATGGAAGTAGAAATGGAAAATTACTTAGTTTAAGGGATGTTCTGGAA
AATATTGGAAGCGGCATAAAAGATAAGAGAAAAAATCAGAGTAAATATACAGATAAATTG
AAAGGGATATTAACAAAAGCAAAATTTGTTGATGGATTGAGTAGTAGATATGGTTATGTA
AGGGATTCTGATGGAATTTCATGTAATCTTAGTCACAAATTCCATACTAATATAACAATT
GAAGCTGCAAGGGATCCTTGTTATGGAAGGGAACAAAACCGITTTGATGAAAATGTCGAA
TCGTATTGTAACAATGATAAAATAAGAGGTAGTGGGAAAATATTTGATGGAAGAGTATGT
GTCCCACCTAGAAGGCAACATATATGTGATCATAATTTAGAATATTTAAATAACAATA
ACTGATGACACTGATGATTTGTTGGGAAATGTGTTAGTTACAGCAAAATATGAAGGTCAA
TCTATTGTTAATAATCATCCACATAAAGAAACTTCTGATGTTTGTACTGCTCTTGCACGA
AGTTTTGCTGATATAGGTGACATTGTAAGAGGAATAGATATGTTTAAACCTAATGACCAA
GACGAAGTATGGAATGGTCTAAGGTCAGTTTTCAAGAAAATACATGATAATTTGTCATCT
GAAGTAAAAAATGCTTATCCAGATGATGGATCTGGAAATTATTTTAAATTAAGGGAAGAT
TGGTGGACAGCGAACAGAGATCAAGTATGGAAAGCCATGACTTGTGTTGCACCAGAAAAT
GCTTATTTTAGAAAAACAGAAGCTGATGGAATAGGAATTTCAAGTTTAATTTTACCATAT
TCTAAATGTGGACGTGATACTGACCCCCCTGTTGTTGATTATATCCCTCAACGCTTAAGA
TGGATGAGTGAATGGTCTGAATATTTCTGTAATGTATTAAATAAAGAAATAGATGAAATG
AATAATCAATGTAAAGATTGTGAAATGAGCCGAAGATGCAATGATGATAGCGAAGGGGGA
AAATGTAAAAAATGCAAAGAACAATGTCAAATATTCAAGGAGCTCGTAAGTAAATGGAAA
AACCAATTTGATAAACAATCAATGAAATATATGGAATTATATAATAAAGCAAGTACTAAT
ATAACTAAACAGAACTCTAGTGCACCTGAACGTGGATATCGACGTAATCATAGACGTAGA
GGTTACGATGATGATACAAATGTACAATTATTTTTGAAAAAAGTAATAGAAAATAATGAG
TGTAAAGTTGAGTCCCTTGGAAAATATCTTGATAAAACAAGTCATTGTGGTAATTATAAT
TTTAATTATGATAATACTCCAGGTTCCAATAGATCTAACGCTTTTGAAATAACTCCAGAA
AAGTTTAAAAAGGCTTGCAAATGTAAAATACCTAATCCATTAGAAAAATGTCCTAATGAA
GAAAACAAAAATGTATGCACAAGATTCGATAAGGTTTATTCATGTACATCACTTTCTTTT
AAAAATGACTTGAGCGAATGGAATAATTCAGGAGTAAAAAATAAAGAAAATGACAATAAT
GGTGTGTTAGTTCCTCCTAGAAGACGAAATTTATGCATAAATTTGTTITCAAAAAAAGAT
TATAAAATGAAAGATGAAAACGATTTCAAAGAGGATCTACTTAATGCTGCTTTTAGTCAA
GGAAAATTGTTAGGAAAGAAATATAGTAACTACAGTAATGAAGCATATGAGGCTATGAAG
TTCAGTTATGCTGATTACAGTGATATCGTGAAAGGTACCGACATGATGAATGATTTAAAA
AAATTAAATAAAGAACTAAATACACTTCTTAAAGAAACTGAAAAAGGAGATATATCTGTG
GATCGTAAAACATGGTGGGATGATAATAAAAATGTTGTATGGAATGCTATGTTATGTGGC
TATAAAACCGAAAATGAAAATCAACAATTGAATTCATCGTGGTGTAATGTACCTGATGAT
GATTATATTGATCAATTTTTGAGATGGTTAACTGAATGGGCCCAACAATATTGTAAAGAA
AAATTAATTAAAGCACATATAATAAATACAAAATGTAAAGATATCGTTGAAGGGAGAAAA
CATAAAAGTATGGTTGATATAACAGATGTAGAATGTAAACGATTATTTATTGATTATGAA
GAATGGTTTCGTTACCGATATAATCAATGGAAGGGATTATCTGAAAAATACATTAAGATT
AAGAAGAGCAAAAATTCTGGAGTGAATATACCCTCTGAGGAATGTGCTGCATCATACGTA
ACAAAACATTGCAATGGATGTATTTGTAATTTGAGAGATATGGAGGATATACATAAAAAC
ATTAATAACCAAAATGAATTAATGAAGGAAATGATTAATATAATTAAATTTGATACTGAT
CAATATAGAACTCAATTACAAAATATATCAAATTCTATGGAAATAAATCCAAAAAGTGTA
AAAACAGCAGTAGATACTACGAAAGATATAGTTTCATATGGATTGGCCGGTACTATGGGA
GTTGCAGCAATTGGATTACAAGCAGGAGATTTTCTTGGAAAAAAAATTCAAGATTTGTAC
AATGAATTTATGAAACCTGTTGAAAAAAAATTAGATACATCATCTAAAAATCTTAATATC
TACGAAGACCCCAACATTATGGTTCCTGCTGGTATTGGTGTCGCCTTAACTCTAGGATTG
TTATTATTTAAGATGAGAAGAAAAGCAAAACGTCAAGTAGATATGATACGGATATTACAA
ATGTCACAAAACGAATATGGAATTCCGACAACCAAATCACCAAACAAATATGTTCCATAT
GGGAGTCAACGATATAAAGGCAAAACATACTTATATGTTGAAGGAGATACAGACGAAGAG
AAATATATGTTTATGTCTGATACTACTGATATAACCTCTTCCGAAAGTGAATATGAAGAA
ATGGATATCAATGATATATATGTTCCTGGTAGTCCAAAATACAAAACGTTGATAGAAGTT
GTTCTGGAGCCATCAAAAAGAGATACACAAAATGATATACCTAGTGATAATACACCTAGT
TATAAACTTACAGATGAGGAATGGAATCAATTGAAAGATGATTTTATATCACAATATTTA
CCAAATACAGAACCAAATAATAATTATAGAAGTGGAAATAGTCCAACAAATACCAATAAT
ACTACCACGTCACATGATAATATGGGAGAAAAACCTTTTATTATGTCCATTCATGATAGA
AATTTATATACTGGAGAAGAAATTAGTTATAATATTAATATGAGTACTAATACTATGGAT
GATCCAAAATATGTATCAAATAATGTATATTCTGGTATTGACCTAATTAATGATTCATTA
AATAGTGGTAATCAACCTATTGATATATATGATGAAGTGCTAAAAAGAAAAGAAAATGAA
TTATTTGGAACAAATCATGTGAAACAAACGAGTATACATAGTGTTGCAAAAAATACATAT
AGTGACGACGCTATAACAAATAAAATAAATTTGTTCCATAAATGGTTAGATAGACATAGA
GATATGTGTGAAAAGTGGGAAAATCATCATGAACGTTTAGCTAAATTAAAAGAAAAATGG
GAAAATGATAATGATGGAGGTAATGTACCTAGTGGTAATCATGTGTTGAATACGGATGTT
TCGATCGAAATAGATATGGATAATCCTAAACCTATAAATCAATTTAGTAATATGGATATA
AACGTGGATACACCTACTATGGATAATATGGAAGATGATATATATTATGATGTAAATGAT
AATGATGATGATAATGATCAACCATCTGTGTATGATATACCTATGGATCATAATAAAGTA
GATGTAGATGTACCTAAGAAAGTACATATTGAAATGAAAATCCTTAATAATACATCTAAT
GGATCGTTGGAACAACAATTTCCTATATCGGATGTATGGAATATATAA
SEQ ID NO: 4
Predicted Protein Sequence
Sequence Length: 1315 aa
MAPKNGSRNGKLLSLRDVLENIGSGIKDKRKNQSKYTDKLKGILTKAKFVDGLSSRYGYV
RDSDGISCNLSHKFHTNITIEAARDPCYGREQNRFDENVESYCNNDKIRGSGKIFDGRVC
VPPRRQHICDHNLEYLNNNNTDDTDDLLGNVLVTAKYEGQSIVNNHPHKETSDVCTALAR
SFADIGDIVRGIDMFKPNDQDEVWNGLRSVFKKIHDNLSSEVKNAYPDDGSGNYFKLRED
WWTANRDQVWKAMTCVAPENAYFRKTEADGIGISSLILPYSKCGRDTDPPVVDYIPQRLR
WMSEWSEYFCNVLNKEIDEMNNQCKDCEMSRRCNDDSEGGKCKKCKEQCQIFKELVSKWK
NQFDKQSMKYMELYNKASTNITKQNSSAPERGYRRNHRRRGYDDDTNVQLFLKKVIENNE
CKVESLGKYLDKTSHCGNYNFNYDNTPGSNRSNAFEITPEKFKKACKCKIPNPLEKCPNE
ENKNVCTRFDKVYSCTSLSFKNDLSEWNNSGVKNKENDNNGVLVPPRRRNLCINLFSKKD
YKMKDENDFKEDLLNAAFSQGKLLGKKYSNYSNEAYEAMKFSYADYSDIVKGTDMMNDLK
KLNKELNTLLKETEKGDISVDRKTWWDDNKNVVWNAMLCGYKTENENQQLNSSWCNVPDD
DYIDQFLRWLTEWAQQYCKEKLIKAHIINTKCKDIVEGRKHKSMVDITDVECKRLFIDYE
EWFRYRYNQWKGLSEKYIKIKKSKNSGVNIPSEECAASYVTKHCNGCICNLRDMEDIHKN
INNQNELMKEMINIIKFDTDQYRTQLQNISNSMEINPKSVKTAVDTTKDIVSYGLAGTMG
VAAIGLQAGDFLGKKIQDLYNEFMKPVEKKLDTSSKNLNIYEDPNIMVPAGIGVALTLGL
LLFKMRRKAKRQVDMIRILQMSQNEYGIPTTKSPNKYVPYGSQRYKGKTYLYVEGDTDEE
KYMFMSDTTDITSSESEYEEMDINDIYVPGSPKYKTLIEVVLEPSKRDTQNDIPSDNTPS
YKLTDEEWNQLKDDFISQYLPNTEPNNNYRSGNSPTNTNNTTTSHDNMGEKPFIMSIHDR
NLYTGEEISYNINMSTNTMDDPKYVSNNVYSGIDLINDSLNSGNQPIDIYDEVLKRKENE
LFGTNHVKQTSIHSVAKNTYSDDAITNKINLFHKWLDRHRDMCEKWENHHERLAKLKEKW
ENDNDGGNVPSGNHVLNTDVSIEIDMDNPKPINQFSNMDINVDTPTMDNMEDDIYYDVND
NDDDNDQPSVYDIPMDHNKVDVDVPKKVHIEMKILNNTSNGSLEQQFPISDVWNI
III - Oligo ID: oPFH0018-P1asmoDB ID: MAL8P1.37
SEQ ID NO: 5
Predicted DNA/mRNA Sequence Sequence Length: 1242 bp
ATGAAAGTCGGAAAATTAAAAAAGAGAAAAAATTCTGGCTTGTTATATCCATATTTTAAG
AATAAATCATTTAGATTAAATAGATATATATTCATAAAACCAATAAAAAGTGTAAAACTA
AATTATAAGAAAAAAAAAATGAACCTAACACATGAAATTTGTATTTTAAATTGTAGTGAA
AAATTAATAGATTATAAACTAGCTTTTCAACTTCAAAATATCCTACATCATTCAAAAATT
ATTATGAAAAATAAAAATGAAGTACAAATATCAAATCATTTAGAATTAAAAAAATTCAAA
AATTTCAAAGAAAACATGGAAAAATATGATTTCTGTTTTATATTACAACATACTCCATGC
TATACCTTAGGTAGTGTAGCAAATTGTAGTGATATACTTCTAGATAAGGAAAATTATTAT
ATTGAAGAATTAGGAGATATATATAATAATTTGTATTCGAATGAAATTATTCATCTTATG
AATAAATGTGAAACAATTCAAGATAAAATTAATCAATCTGATATATATAATGAAAATACA
AATTATTTCAATAATTTTTTAAAACATTGTAGACAAAGAAAAATACCCATTTATCGAGTT
AACAGGGGAGGCAAAGCTACATACCATGGACCTGGACAGTTAGTATTATATTTTATATTT
AACTTAAAAAATTATCCATCCAATTATAATGAGCGAATTATAAATAAGCACTATAAATAT
ACAAACAAAGAAAACTTTCCATCAAAAACATCGGAATATGAAAAAAATAACATATATACA
AATTCAAACAGTAAAGAAAACATATCATCTATAGAACGCACTTTTGATTTGCGCACAACA
ATAAATAACTTTCAAAAAATTGGAATGGAAACCTTGCAAAAATTTAATATAAAAACACAC
TGTAAAAAAGATACAATAGGTATCTTTTATAAGGATAAAAAAATTATATCCATAGGATTG
AAAATAACAAAATATATATCTATGCATGGATTGTCATTAAATTTTAATCTCGATAACAAT
TTTTTAAAATATCTATTATCATGTGGTATGAATCATAATGATTATATATCCATGCATGAA
ATAAATGAAATGAAAAAAAAAAATTATATTTATCAAAAAGGAAAAATAGCTAGTAGCTCA
AATATATTAAATGAATTAACTTTAAATATAACAGAGTCATTAAAAAAGGTGTTTAATGTA
AAAGTAAGAAATATAAAAGATATACGAGAAATGTTTTATTAA
SEQ ID NO: 6
Predicted Protein Sequence
Sequence Length: 413 aa
MKVGKLKKRKNSGLLYPYFKNKSFRLNRYIFIKPIKSVKLNYKKKKMNLTHEICILNCSE
KLIDYKLAFQLQNILHHSKIIMKNKNEVQISNHLELKKFKNFKENMEKYDFCFILQHTPC
YTLGSVANCSDILLDKENYYIEELGDIYNNLYSNEIIHLMNKCETIQDKINQSDIYNENT
NYFNNFLKHCRQRKIPIYRVNRGGKATYHGPGQLVLYFIFNLKNYPSNYNERIINKHYKY
TNKENFPSKTSEYEKNNIYTNSNSKENISSIERTFDLRTTINNFQKIGMETLQKFNIKTH
CKKDTIGIFYKDKKIISIGLKITKYISMHGLSLNFNLDNNFLKYLLSCGMNHNDYISMHE
INEMKKKNYIYQKGKIASSSNILNELTLNITESLKKVFNVKVRNIKDIREMFY
IV - OligoID: F44947_3 - PlasmoDB ID: PFD0235c
SEQ ID NO: 7
Predicted DNA/mRNA Sequence Sequence Length: 1704 bp
ATGAGAAGGTACCTGTTGATTACCTGTTTGTTTGTCCTGTGTTGCTTAAAATTAAAGCAT
GTGAACTTTTTAAAGTGGGAGCAGGAAAATGATTTTTATTATATAAATAATGAGAAACTA
TTAAAAAGGGTATTACATAATGTAGAACAAACTAAAGAAAGAACAGAAGTTGATAAACCA
ATAGTATTTGGTATAAGGAAAGGAAAATTTGTTACAATACACAAAGAAACAAAAGAAGAG
AAGATGCTGAAGGATAATTTGATAGAAGCTATATTATTTGATCCTAAGAAAGATGAAGAA
TTAAAAATTGATATAAAAGAAACAAATATAGATAAAGATAGAAAAAAAAATCAAAAAAGA
GAAAATGGAATTATTAAAGATGATACAGCTAAGGATAAGGATTTGTATTCATATACTAAA
GACCCGATTACTCTCCATAAAAAAAAATTAAAAGAAGAAAAGAATTTTGTTATGATCAAA
GAATTTGTAAAAGATTTATCTAGTCGAGATGAAAATGTATTAATATCTAATGTGAACATT
TTTTTAAAAAGAATATTTAATTTGATATTGAGGGAAAAAATAATTACTGCAATGTGTTCA
GATGTACAAAATGAAGGAATAGAAAATAATAACACACAAATGAAGGGCAAACAAATAAAG
GACGCACAAATGAAGGGCAAACAAAATAATAACACACAAATGAAGGGCAAACAAAATAAT
AACACACAAATGAAGGGCAAACAAAATAATAACACACAAATGAATGACGCACAAATGAAT
GACGCACAAAATTATGATGGCAAAGATAACAATTCAGAATGCTTGAAAAATAATAAGAAT
TGTAATTTCGATAACAAAATCAAGATTAAAGATTGTAGTAAGGGTTCCATAAGTTGTTTT
CTCTCGAACATTAAAAATGAAGAATTTTATAAAGCTCCAGATTTATTTAAATATTATATA
TCTTTAGAAAAAATGTTGAGGAGCTCTTCTGITCGATCCAAAACAGACAGGATATCAAAA
TATTTTACTTTTTATCCAGTATCTATGGATAAAGAATATTATGAAGAGAAAATAAATAAT
CATGTATTTTTAGAGGCTGTTAGAAATATATTATTTGATTTAGATGAAGGAAATAAAAAG
GATAAAAAAAAGGTTTTTTCGAGTTTIGTAATAGTCGTAGATACATTAATATCTTTAATA
AAAAAAGAAAAGGTAGTAAAAGAAATGTATATGTTTATACATTTATTTTTTCAAGATTTA
AATTTATTAAATAAAAAAATATTAGACATTTTATTAAAAAGTTCTTTTAAGCCAGGAGCA
TCATTTAATATTCCAGATTTCAATAAGAAAAATTTCGAATTTATTTTATCAAGAATATAT
ACAAGATATGTTTTAAATAATTTATTAAATAAGACATTCAATAATTCAGATACCATCAAT
ATGTCTGATTTTTTAAATAACAAAATAAAACCTTTCAATTTTAGTTTTACGGAAACAAGT
GTAAACTTGCTAAAGAATGAGGGTATTCAGATAAAGGATGATGACCTTTTGGTGAGCGAA
GAAAATTTGTGTAAATATATACCTATCAAAAAAAAATTATTATATGAAAAACTTAACAAG
ACAAGGAAAGCTGCAGAGGAAGCTATACTGGATTATATATTTAGACTTTTATTAAGAAAA
TTACATGAATTTATAACAGAATAA
SEQ ID NO: 8
Predicted Protein Sequence
Sequence Length: 567 aa
MRRYLLITCLFVLCCLKLKHVNFLKWEQENDFYYINNEKLLKRVLHNVEQTKERTEVDKP
IVFGIRKGKFVTIHKETKEEKMLKDNLIEAILFDPKKDEELKIDIKETNIDKDSKKKQKR
ENGIIKDDTAKDKDLYSYTKDPITLHKKKLKEEKNFVMIKEFVKDLSSRDENVLISNVNI
FLKRIFNLILREKIITAMCSDVQNEGIENNNTQMKGKQIKDAQMKGKQNNNTQMKGKQNN
NTQMKGKQNNNTQMNDAQMNDAQNYDGKDNNSECLKNNKNCNFDNKIKIKDCSKGSISCF
LSNIKNEEFYKAPDLFKYYISLEKMLRSSSVRSKTDRISKYFTFYPVSMDKEYYEEKINN
HVFLEAVRNILFDLDEGNKKDKKKVFSSFVIVVDTLISLIKKEKVVKEMYMFIHLFFQDL
NLLNKKILDILLKSSFKPGASFNIPDFNKKNFEFILSRIYTRYVLNNLLNKTFNNSDTIN
MSDFLNNKIKPFNFSFTETSVNLLKNEGIQIKDDDLLVSEENLCKYIPIKKKLLYEKLNK
TRKAAEEAILDYIFRLLLRKLHEFITE

### DETAILED DESCRIPTION OF THE INVENTION

Radiation attenuated sporozoites are metabolically active. They can invade human hepatocytes in vitro, and develop into early liver stage parasites. However, they are not capable of completing maturation in hepatocytes, and thus cannot develop into the erythrocytic stages of P. falciparum malaria that cause clinical disease. Therefore, the protective immune response induced by immunization with radiation attenuated P. falciparum sporozoites has to be directed against proteins expressed in sporozoites or these early liver stage parasites. This then is the rationale for identifying those sporozoite transcripts that are differentially regulated in radiation attenuated sporozoites, and utilizing the corresponding encoded proteins in the preparation of subunit vaccines.

### DEFINITIONS

“Conferring protective immunity” as used herein refers to providing to a population or a host (e.g., an individual) the ability to generate an immune response to protect against a disease (e.g., malaria) caused by a pathogen (e.g., Plasmodium falciparum) such that the clinical manifestations, pathology, or symptoms of disease in a host are prevented or substantially reduced as compared to a non-treated host, or such that the rate at which infection, or clinical manifestations, pathology, or symptoms of disease appear within a population are reduced by at least 50%, as compared to a non-treated population when challenged by the pathogen subsequent to the administration of a vaccine. Symptoms of malaria include fever and flu-like illness, including shaking chills, headache, muscle aches, and tiredness. Nausea, vomiting, and diarrhea may also occur. Malaria may cause anemia and jaundice (yellow coloring of the skin and eyes) because of the loss of red blood cells. Infection with one type of malaria, Plasmodium falciparum, if not promptly treated, may cause kidney failure, seizures, mental confusion, coma, and death. Malaria is confirmed by peripheral blood smears looking for the Plasmodium parasite as well as the rapid malaria antigen test, both well known in the art (See The Merck Manual, Eighteenth Edition, Merck and Company 2006, Whitehouse Station, N.J.).

“Immune response” as used herein means a response in the recipient to the introduction of attenuated sporozoites generally characterized by, but not limited to, production of antibodies and/or T cells. Generally, an immune response may be a cellular response such as induction or activation of CD4+ T cells or CD8+ T cells specific for Plasmodium species epitopes, a humoral response of increased production of Plasmodium-specific antibodies, or both cellular and humoral responses. With regard to a malaria vaccine, the immune response established by a vaccine comprising sporozoites includes but is not limited to responses to proteins expressed by extracellular sporozoites or other stages of the parasite after the parasites have entered host cells, especially hepatocytes and mononuclear cells such as dendritic cells and/or to components of said parasites. In the instant invention, upon subsequent challenge by infectious organisms, the immune response prevents development of pathogenic parasites to the asexual erythrocytic stage that causes disease.

“Mitigate” as defined herein means to substantially reduce, or moderate in intensity, symptoms and pathology of malaria which might manifest subsequent to vaccination.

“Replicon” is a genetic unit of replication comprising a length of DNA and a site for initiation of replication. As used herein, a replicon would include a DNA coding sequence operably linked to an initiation sequence or in an expression vector such as a plasmid or a phage.

“Therapeutic” as defined herein relates to reduction of clinical manifestations or pathology which have already become manifest. A “therapeutically effective amount” as used herein means an amount sufficient to reduce the clinical manifestations, pathology, or symptoms of disease in an individual, or an amount sufficient to decrease the rate at which clinical manifestations, pathology, or symptoms of disease appear within a population.

“Vaccine” as used herein is a preparation comprising an immunogenic agent and a pharmaceutically acceptable diluent in combination with excipient, adjuvant, additive and/or protectant. The immunogen may be comprised of a whole infectious agent or a molecular subset of the infectious agent (produced by the infectious agent, synthetically or recombinantly). When the vaccine is administered to a subject, the immunogen stimulates an immune response that will, upon subsequent challenge with infectious agent, protect the subject from illness or mitigate the pathology, symptoms or clinical manifestations caused by that agent. The vaccine, according to the invention, can be either therapeutic or prophiylactic. A therapeutic (treatment) vaccine is given after infection and is intended to reduce or arrest disease progression. A preventive (prophylactic) vaccine is intended to prevent initial infection or reduce the burden of the infection. Agents used in vaccines against a parasitic disease such as malaria may be whole-killed (inactive) parasites, live-attenuated parasites (unable to fully progress through their life cycle), or purified or artificially manufactured molecules associated with the parasite—e.g., recombinant proteins, synthetic peptides, DNA plasmids, and recombinant viruses or bacteria expressing Plasmodium proteins. A vaccine may further comprise sporozoites along with other components such as excipient, diluent, carrier, preservative, adjuvant or other immune enhancer, or combinations thereof, as would be readily understood by those in the art.

### Preparation of Sporozoites

Plasmodium-species parasites are grown aseptically in cultures as well as in vivo in Anopheles-species mosquito hosts, most typically Anopheles stephensi hosts. Methods of growing Plasmodium-species parasites, particularly, methods of growing sporozoites in mosquitoes, are known in the art and have been described (See, Hoffman & Luke, U.S. Pat. No. 7,229,627; US Pub. No. 2005/0220822; and Munderloh, U. G. and T. J. Kurti (1985) Experietia 41:1205-1207).

Mosquitoes are infected with the species of Plasmodium to be analyzed. For example, in a preferred embodiment, to analyze P. falciparum, mosquitoes are infected with the NF54 strain P. falciparum. Infected mosquitoes are divided into two groups. One group receives a dosage of radiation sufficient to attenuate the parasites such that are still capable of infecting the host, but not capable of developing beyond liver stage, typically 150 Gy of gamma irradiation from a ⁶⁰Co source (irradiated). The second group is not exposed to radiation (non-irradiated). Sporozoites are isolated from mosquito salivary glands and purified.

Attenuation: Methods of attenuating sporozoites by radiation exposure have been disclosed (See, e.g., Hoffman & Luke, U.S. Pat. No. 7,229,627) and are incorporated herein by reference. Sporozoites may be attenuated by at least 100 Gy but no more than 1000 Gy, preferably 120 to 200 Gy, and most preferably about 150 Gy. The attenuation of Plasmodium parasites of the vaccine disclosed herein allow sporozoite-stage parasites to remain metabolically active, infectious with the ability to invade hepatocytes (potency); while ensuring that parasites do not develop to the fully mature liver schizont stage, cannot reenter the host bloodstream, invade erythrocytes or reach the developmental stages which cause disease (safety). Those of skill in the art can routinely determine the developmental stages of the parasite and adjust the attenuation as necessary.

To isolate both the irradiated and non-irradiated sporozoites, salivary glands from 150 to 400 mosquitoes of each, for example, are dissected and separately pooled. The sporozoites are released from the salivary glands by passage back and forth in a needle and syringe (trituration). Sporozoites are pooled in an excipient, typically one percent human serum albumin (HSA) in Medium 199 with Earle's salts (E-199).

### Identification of Differentially Expressed Genes in Irradiated Sporozoites

To identify the genes and proteins that contribute to protection, RNA isolated from infectious sporozoites harvested from salivary glands of mosquitoes after irradiation attenuation can be compared with RNA isolated from non-irradiated infectious sporozoites harvested from mosquito salivary glands. The RNA can be first reversed transcribed to cDNA. It can then be coupled to different ester dyes separately to generate reagents that are used as probes of a microarray representing the entire ˜5,400 genes in the P. falciparum genome.

Separate experimental batches of RNA can be prepared from different lots of P. falciparum sporozoites (irradiated and non-irradiated). Because of the low total RNA yields from sporozoites, an amplification step can be required before array hybridization. To maximize confidence in the findings a number of steps in the process can be included to filter out potentially poor quality results. First the RNA samples from the 3 experimental batches can be assessed on a number of different arrays. Second, only data from those spots with rQuality scores >0.5 can be included in the analyses. Third, only arrays that have an extremely high correlation between technical replicates (r²>0.80) can be used in the final analysis. Genes that are up- or down-regulated in all arrays with this high level of correlation can be considered.

Good reproducibility between technical replicate arrays is important. Irradiation of sporozoites can identify a number of up- or down-regulated genes, typically about 100-250 genes in any given experiment. Generally, differential changes in expression can be low (1.5-2.5 fold). Those genes up- or down-regulated at statistically significantly levels can be analyzed further and selected as targets for malarial subunit vaccine development.

In one aspect of the invention, the differentially expressed gene product corresponds to PFC0166w, a redox-active protein in P. falciparum, named plasmoredoxin (Plrx), which is highly conserved but found exclusively in malarial parasites. The gene does not have introns, therefore both the DNA and RNA sequences are identified herein as SEQ ID NO:1 and the encoded polypeptide sequence is identified herein as SEQ ID NO:2.

In a further aspect of the invention, the differentially expressed gene product corresponds to PFI1820w a member of the variant (var) gene family that plays a role in adhesion of infected erythrocytes to endothelial cells in the brain and other organs, and is involved in immune evasion. The gene does not have introns, therefore both the DNA and RNA sequences are identified herein as SEQ ID NO:3 and the encoded polypeptide sequence is identified herein as SEQ ID NO:4.

In a further aspect of the invention, the differentially expressed gene product corresponds to MAL8P1.37 a putative lipoate-protein ligase in irradiated sporozoites, which may play an important role in the metabolism and survival of these attenuated forms in hepatocytes. The gene does not have introns, therefore both the DNA and RNA sequences are identified herein as SEQ ID NO:5 and the encoded polypeptide sequence is identified herein as SEQ ID NO:6.

In a further aspect of the invention, the differentially expressed gene product corresponds to PFD0235c, a conserved hypothetical protein. The gene does not have introns, therefore both the DNA and RNA sequences are identified herein as SEQ ID NO:7 and the encoded polypeptide sequence is identified herein as SEQ ID NO:8.

In further aspects, the protein of the invention can comprise, or alternatively consist of, an amino acid sequence which is at least 80%, 85%, 90%, 95%, 96%, 97%, 98%, 99% identical to the polypeptide sequence of SEQ ID NO:2, SEQ ID NO:4, SEQ ID NO:6, or SEQ ID NO:8.

Each of the differentially expressed proteins may individually or in combination result in protective immunity. Therefore, in a set of embodiments, each of the polypeptides products of the four genes identified herein, individually or in combinations, is used in subunit recombinant protein vaccines.

In further aspects, a polypeptide of the invention can comprise, or alternatively consist of, an immunogenic fragment of the polypeptide sequence of SEQ ID NO:2, SEQ ID NO:4, SEQ ID NO:6, or SEQ ID NO:8. In particular, an immunogenic fragment can comprise, consists essentially of, or consist of at least about four to five amino acids of SEQ ID NO:2, SEQ ID NO:4, SEQ ID NO:6, or SEQ ID NO:8, at least seven, at least nine, or between at least about 15 to about 30 amino acids of SEQ ID NO:2, SEQ ID NO:4, SEQ ID NO:6, or SEQ ID NO:8. The amino acids of a given epitope of SEQ ID NO:2, SEQ ID NO:4, SEQ ID NO:6, or SEQ ID NO:8 as described may be, but need not be contiguous or linear. In certain other embodiments the immunogenic fragment comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:2, SEQ ID NO:4, SEQ ID NO:6, or SEQ ID NO:8, where non-contiguous amino acids form an epitope through protein folding.

### Use of Gene Products in Malarial Vaccines

The DNA sequences and proteins disclosed herein are useful as vaccines and vaccine components which provide partial, enhanced, or full protection of human subjects who have not previously been exposed to a malaria-causing pathogen, or have been exposed, but are not fully protected. The materials and methods disclosed may also be used to reduce the chance of developing a Plasmodium infection, reduce the chance of becoming ill when one is infected, reduce the severity of the illness, such as fever, when one becomes infected, reduce the concentration of parasites in the infected person, or to reduce mortality or morbidity from malaria when one is exposed to malaria parasites. In many cases even partial protection is beneficial. For example, a vaccine treatment strategy that results in any of these benefits of about 30% of a population may have a significant impact on the health of a community and of the individuals residing in the community.

Methods of DNA synthesis are also well known in the art. See, e.g. Uhlmann E. (1988) Gene. November 15; 71(1):29-40. Applicant's experience with expression of proteins in Pichia pastoris indicates that altering the codon usage (the degenerative flexibility of the DNA sequence code means multiple triplets code for the same amino acid) leads to enhanced expression of the protein (Narum, D L, et al, Codon Optimization of Gene Fragments Encoding Plasmodium falciparum Merozoite Proteins Enhances DNA Vaccine Protein Expression and Immunogenicity in Mice. (2001) Infection and Immunity 69:7250-7253, incorporated in its entirety herein by reference). Furthermore, this alteration of codon usage also enhances in vivo expression of proteins in DNA vaccines. We therefore used native Pv CSP encoding sequences as the basis for creating synthetic genes in which the sequence has been altered to optimize codon usage.

The present invention encompasses the use of variants of DNA sequences as described. Variants may contain alterations in the coding regions, non-coding regions, or both. Examples are polynucleotide variants containing alterations which produce silent substitutions, additions, or deletions, but do not alter the properties or activities of the encoded polypeptide. In certain embodiments, nucleotide variants are produced by silent substitutions due to the degeneracy of the genetic code. In further embodiments, polynucleotide variants can be produced for a variety of reasons, e.g., to optimize codon expression for a particular host. Accordingly, the present invention encompasses an isolated polynucleotide as described above comprising a nucleic acid that is 70%, 75%, or 80% identical, at least about 90% to about 95% identical, or at least about 96%, 97%, 98%, 99% or 100% identical to SEQ ID NOs: 1, 3, 5 or 7.

Methods for expressing encoded polypeptides in a DNA plasmid, recombinant virus, recombinant bacteria, replicon, or other DNA or RNA based vaccine delivery system, or to produce a recombinant protein or synthetic peptide are well known in the art.

As a vaccine, the Plasmodium proteins and synthetic peptide are preferably delivered with adjuvant. Any adjuvant may be used. The most commonly used human adjuvants include mineral salts (e.g., aluminum hydroxide and aluminum or calcium phosphate gels). Another class which includes adjuvants approved for human use is oil emulsions and surfactant based formulations (e.g., MF 59, QS 21, AS 02, and Montanide ISA-51 and ISA-720). Other classes include particulate adjuvants (e.g., virosomes, AS 04, and ISCOMS; microbial derivatives (natural and synthetic) (e.g., monophoshoryl lipid A, Detox, AGP, DC Chol, OM-174, CpG motifs, modified LT and CT; endogenous human immunomodulators (e.g., hGM-CSF and hIL-12, Immudaptin; and finally inert vehicles such as gold particles.

### Methods of Prevention and Treatment of Malaria

Also disclosed are methods for prevention and treatment of malaria in a subject, which methods comprise administering to the subject an amount effective to treat or prevent malaria of a vaccine comprising the novel compositions disclosed herein. The subject to which the vaccine is administered in accordance with these methods may be any human or non-human animal susceptible to infection with the malaria parasite. For such methods, administration can be oral, parenteral, intranasal, intramuscular, or any one or more of a variety of well-known administration routes other than intravenous. Moreover, the administration may be by continuous infusion or by single or multiple boluses.

In an embodiment, the vaccine is administered intramuscularly in the deltoid in a 3 dose regimen, given at 20 to 60 day intervals. The volume of vaccine delivered is 0.1 to 1.0 ml, preferably 0.5 ml, and the amount of immunogen is 25 to 75 micrograms, preferably 50 micrograms.

The effectiveness of treatment of malaria may be readily ascertained by the skilled practitioner by evaluation of infection in red blood cells (erythrocytes) or clinical manifestations associated with malarial infection, for example fatigue, headache, elevated temperature, and coma. Thus a subject with a Plasmodium infection and symptoms of malaria shows improved or absent clinical manifestations of malaria infection following administration of the novel compositions disclosed herein.

The prevention of malaria by methods disclosed herein is measured by the percent reduction of Plasmodium blood-stage infection and/or clinical manifestations of disease in subjects upon subsequent challenge with or exposure to infectious Plasmodium parasites of the same species from which the subject material of the invention was derived.

Generating an immune response in a subject can be measured by standard tests of humoral and cellular immunity including, but not limited to, the following: direct measurement of peripheral blood lymphocytes by means known to the art; natural killer cell cytotoxicity assays (Provinciali et al. (1992) J. Immunol. Meth. 155: 19-24), cell proliferation assays (Vollenweider et al. (1992) J. Immunol. Meth. 149: 133-135), immunoassays of immune cells and subsets (Loeffler et al. (1992) Cytom. 13: 169-174; Rivoltini et al. (1992) Can. Immunol. Immunother. 34: 241-251); interferon gamma ELIspot assays, and skin tests for cell mediated immunity (Chang et al. (1993) Cancer Res. 53: 1043-1050). For an excellent text on methods and analyses for measuring the strength of the immune system, see, for example, Coligan et al. (Ed.) (2000) Current Protocols in Immunology, Vol. 1, Wiley & Sons. Therapeutically effective amounts of the compositions are provided as vaccines. The vaccines comprise therapeutically effective amounts of recombinant proteins, synthetic polypeptides, recombinant viruses, recombinant bacteria, recombinant parasites, DNA or RNA encoding recombinant or synthetic polypeptides or combinations of DNA and polypeptides as well as attenuated Plasmodium species (particularly including P. vivax or P. falciparum) sporozoites, as components in a regimen.

Methods of administration of peptide, protein, recombinant viruses, bacteria, and parasite, DNA or RNA vaccines are known in the art. As used herein, the term “administration” or “administering” refers to the process of delivering an agent to a subject, wherein the agent directly or indirectly increases the titer of immune response within the subject to appropriate Plasmodium-specific blood antigens. The process of administration can be varied, depending on the agent, or agents, and the desired effect. Thus, the process of administration involves administering a selected immunogen to a patient in need of such treatment. Administration can be accomplished by any means appropriate for the therapeutic agent, for example, by parenteral, mucosal, pulmonary, topical, catheter-based, or oral means of delivery. Parenteral delivery can include for example, subcutaneous, intradermal, intravenous, intramuscular, intra-arterial, and injection into the tissue of an organ. Mucosal delivery can include, for example, intranasal delivery, preferably administered into the airways of a patient, i.e., nose, sinus, throat, lung, for example, as nose drops, by nebulization, vaporization, or other methods known in the art. Pulmonary delivery can include inhalation of the agent. Oral delivery can include delivery of a coated pill, or administration of a liquid by mouth. Administration can generally also include delivery with a pharmaceutically acceptable carrier, such as, for example, a buffer, a polypeptide, a peptide, a polysaccharide conjugate, a liposome, and/or a lipid, according to methods known in the art.

Compositions of the subject invention can be formulated according to known methods for preparing pharmaceutically useful compositions. Formulations are described in a number of sources which are well known and readily available to those skilled in the art. For example, Remington's Pharmaceutical Science (Martin EW [1995] Easton Pa., Mack Publishing Company, 19th ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The foimulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

Therapeutically effective and optimal dosage ranges for vaccines and immunogens can be determined using methods known in the art. Guidance as to appropriate dosages to achieve an anti-viral effect is provided from the exemplified assays disclosed herein. More specifically, results from the immunization pattern described herein can be extrapolated by persons having skill in the requisite art to provide a test vaccination schedule. Volunteer subjects or test animals can be inoculated with varying dosages at scheduled intervals and test blood samples can be evaluated for levels of antibody and/or sporozoite neutralizing activity present in the blood, using the guidance set forth herein for evaluation of rabbit blood. Such results can be used to refine an optimized immunization dosage and schedule for effective immunization of mammalian, specifically human, subjects.

Methods of formulating pharmaceutical compositions and vaccines are well-known to those of ordinary skill in the art (See, e.g., Remington's Pharmaceutical Sciences, 18^{th} Edition, Gennaro, ed. (Mack Publishing Company: 1990)). Such vaccines may be for administration by oral, parenteral (intramuscular, intraperitoneal, or subcutaneous injection), transdermal (either passively or using iontophoresis or electroporation), transmucosal (nasal, vaginal, rectal, or sublingual) routes of administration or using bioerodible inserts and can be formulated in dosage forms appropriate for each route of administration. In general, vaccines herein comprise recombinant or synthetic components of Plasmodium sporozoites, together with pharmaceutically acceptable carriers. Such compositions include diluents of various buffer content (e.g., Tris-HCl, acetate, phosphate), pH and ionic strength; anti-oxidants (e.g., ascorbic acid, sodium metabisulfite), and preservatives (e.g., Thimersol, benzyl alcohol); incorporation of the material into particulate preparations of polymeric compounds such as polylactic acid, polyglycolic acid, etc. or into liposomes. See, e.g., Remington's Pharmaceutical Sciences, 18th Ed. (1990, Mack Publishing Co., Easton, Pa. 18042) pages 1435-1712 which are herein incorporated by reference.

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets, pellets, powders, or granules. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include the therapeutic agent and inert ingredients which allow for protection against the stomach environment, and release of the biologically active material in the intestine.

Vaccines for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants, preserving, wetting, emulsifying, and dispersing agents. They can also be manufactured using sterile water, or some other sterile injectable medium, immediately before use.

In order to determine the effective amount of the vaccines, the ordinary skilled practitioner, considering the therapeutic context, age, and general health of the recipient, will be able to ascertain proper dosing. The selected dosage depends upon the desired therapeutic effect, on the route of administration, and on the duration of the treatment desired. The dosing schedule may vary, depending on the circulation half-life and the formulation used. Approaches to determine levels for dosages are known in the art. Animal models of malaria are known to those in the art. These include non-human primates, of which one used for P. vivax and P. falciparum is the Aotus monkey (Jones et al. (2000) Am. J. Trop. Med. Hyg., 62: 675-680).

Vaccines may be administered in conjunction with one or more additional active ingredients, pharmaceutical compositions, or vaccines.

The pharmaceutical composition may be preserved, cryopreserved, lyophilized, refrigerated, or the like. A kit may additionally comprise carrier, either in combination with or separate from the pharmaceutical composition. A kit may additionally comprise means for delivery of the pharmaceutical composition, such as syringe and needle or microneedle, or alternatively, any of the means for delivery provided in the instant specification.

Disclosed vaccines and disclosed methods of using these vaccines may be useful as separate elements of a vaccine regimen, each in turn comprising a discrete vaccine to be administered separately to a subject. Regimens may include prime/boost, preferably combining Plasmodium-related DNA vaccine or recombinant virus comprising Adenovirus as a prime and polypeptide vaccine as a boost. A vaccine complex comprising separate components may be referred to as a vaccine regimen, a prime/boost regimen, component vaccine, a component vaccine kit or a component vaccine package, comprising separate vaccine components. For example, a vaccine complex may comprise as a component one or more recombinant or synthetic subunit vaccine components disclosed herein, including but not limited to recombinant protein, synthetic polypeptide, DNA encoding these elements per se or functionally incorporated in recombinant virus, recombinant bacteria, or recombinant parasite. Another vaccine component may comprise one or more of these compositions or Plasmodium-related native DNA, native protein, or attenuated or recombinant sporozoites or sporozoite DNA, or RNA—from the same or other Plasmodium species.

### EXAMPLES

The invention now being generally described, it will be more readily understood by reference to the following examples, which are included merely for purposes of illustration of certain aspects and embodiments of the present invention, and are not intended to limit the invention.

### Example 1

### Identification of Differentially Expressed Transcripts in Radiation-Attenuated P. falciparum Sporozoites

Transcript profiles for irradiated and non-irradiated P. falciparum sporozoites were compared in an attempt to identify transcripts that are reproducibly differentially expressed by irradiation (150 Gy). Twenty 70-nucleotide oligo-arrays representing the complete P. falciparum genome (7,256 parasite oligo probes) were separately hybridized with cDNA obtained from 3 different preparations of irradiated and non-irradiated sporozoites isolated from mosquito salivary glands. Of these twenty (20), eight (8) arrays were chosen for further analysis based on r² correlation analyses (r²>0.80) between arrays within an experimental preparation batch. For each array sample, differentially regulated genes were selected by screening based on 1) fold change (>1.5 fold—both up and down regulated) and also 2) rQuality which scores the signal quality from each spot on the array (rQ>0.5). By this method, approximately 60-80 up-regulated loci and 50-150 down-regulated loci were found in common among array samples within an experimental batch. These shared responders from each of the three experimental batches were then compared, in order to determine which genes were consistently affected by irradiation. At this level of stringent selection, 4 loci were identified with high confidence (SEQ ID NOs: 1, 3, 5, and 7). Three transcripts, identified as SEQ ID NOs: 1, 3, and 5, were derived from 3 different gene families, and the fourth, SEQ ID NO:7, from a conserved hypothetical protein of unknown function. All four loci are up-regulated in radiation attenuated sporozoites. These four genes do not have introns, therefore, the sequences provided are the DNA and RNA sequences.

### Preparation of RNA

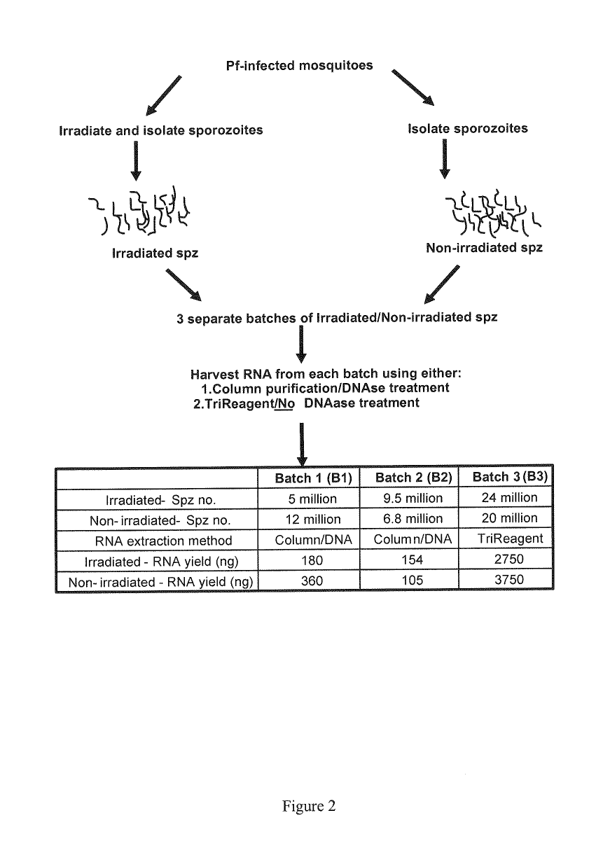

RNA was harvested from sporozoites in 3 separate paired batches or irradiated and non-irradiated sporozoites using either a column purification method followed by DNAse treatment (High Pure RNA Isolation Kit (Catalogue # 11 828 665 001), Roche Applied Science) or Tri-Reagent ® (Cat. No. TR 118, Method 2.2, Molecular Research Center). The latter did not involve DNAse treatment. In a preferred embodiment, the High Pure RNA isolation kit is used. Sporozoite and total RNA yields from all three batches are provided in FIG. 2.

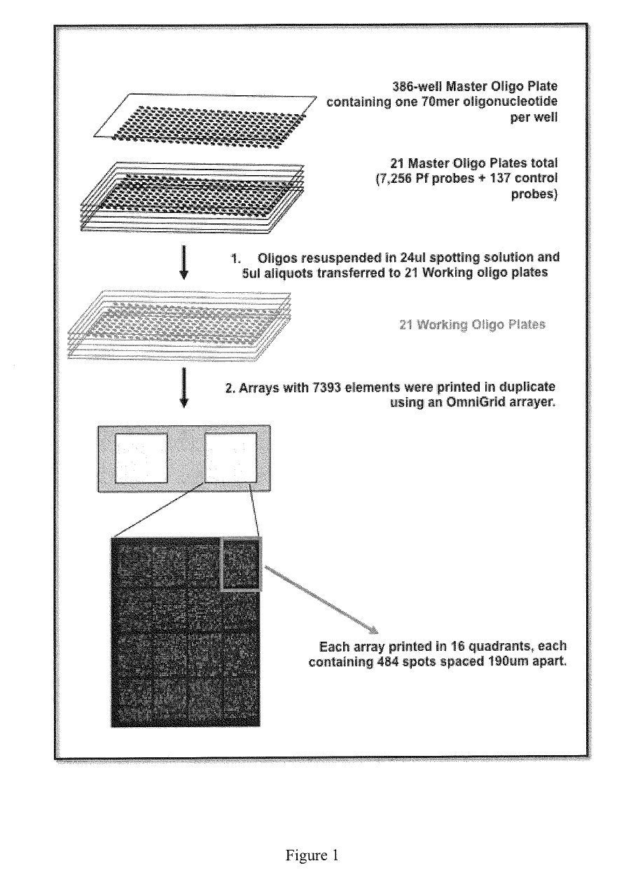

Micro-array Fabrication: As shown schematically in FIG. 1, a set of 7393, 70-mer oligonucleotides (malaria v1.1) developed by Joseph DeRisi's group and used to construct a first generation P. falciparum micro-array (5) representing all 5,400 (approximately) predicted ORFs (6) in the genome, was purchased from Operon Biotechnologies. The entire set of oligos was manually resuspended in 1:1 spotting solution (Pronto Universal spotting solution, Corning) in 21 386-well plates and manually aliquoted into working plates. Arrays were printed in duplicate on poly-L-lysine coated glass slides using an OmniGrid arrayer (GeneMachines, San Carlo, Calif.). The arrayer employs 16 pins in a 4×4 configuration, with each pin programmed to print 484 spots spaced 190 um apart from each other in a 22×22 configuration. Each spot represents a unique oligo from the entire set of 7256 parasite-specific probes and 137 control oligos.

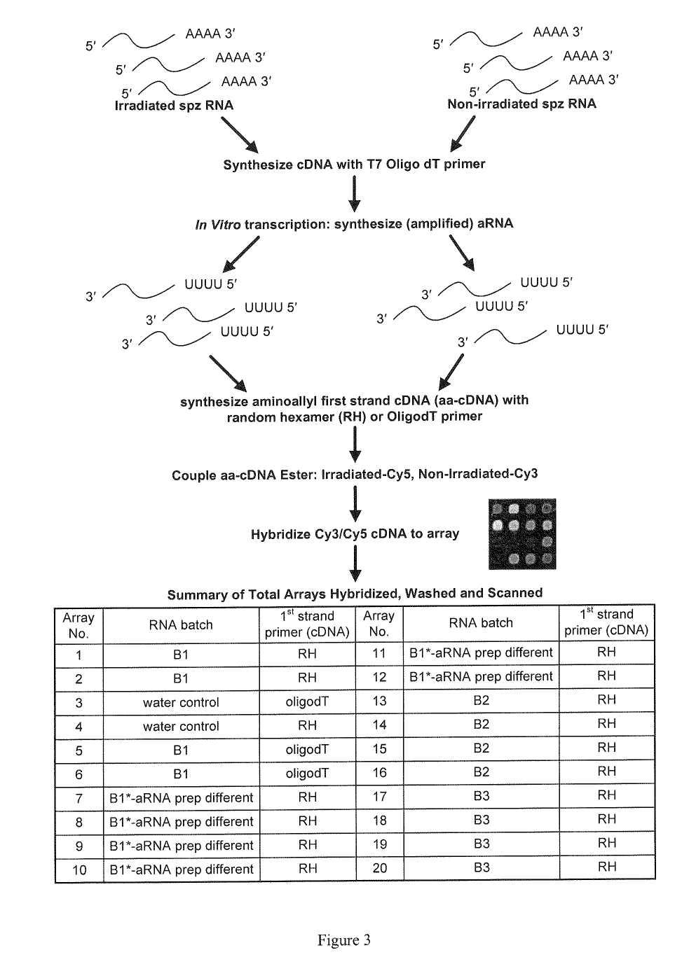

cDNA Synthesis Labeling and Hybridization: Methods of probe labeling and purification, hybridization and array scanning were performed as described (C.C. Xiang, et al. (2002) Nature Biology 20:738-42). As shown in FIG. 3, RNA from each sample was amplified. 100-500 ng of total RNA was reverse transcribed using T7 oligo-dT primer. The resulting cDNA was transcribed in vitro, yielding ˜30-100 ug of amplified RNA (aRNA). 1-2 ug of aRNA can be primed with random hexamer primers (RH) or oligo-dT primer (together with aminoallyl-dUTP) to synthesize aminoallyl first strand cDNA (aa-cDNA). In a preferred embodiment, RH primers are used as probes primed with RH primers gave better results than those primed with oligo-dT. Monofunctional NHS-ester dyes (Cy3 and Cy5, Amersham) are coupled to aa-cDNA (7). Probes from the irradiated sample (experimental) were labeled with Cy5 (fluoresces red) and those from the non-irradiated sample (reference) were labeled with Cy3 (fluoresces green). Cy5/Cy3 probes were combined and hybridized onto each array at 42° C. overnight. Slides were washed and read using a GenePix 4000A scanner (Axon, Foster City, Calif.) at 10 μm resolution. Photomultiplier tube voltage settings are varied to obtain maximum signal intensities with <1% probe saturation. Resulting TIFF images were analyzed with IPLab software (Fairfax, Va.). The Cy5/Cy3 ratios of experimental sample intensities to reference intensities for all targets were computed, and then ratio normalization performed to set the center of the ratio distribution to 1.0 (8). To assess reliability of each ratio measurement, a quality score ranging from 0 (low) to 1 (high) was determined for each spot location (termed rQuality). A summary of the 20 scanned arrays is shown in FIG. 3. Technical replicates within an experimental batch were color coded as follows: arrays 1,2,5,6 in pink (BI); arrays 7-12 in purple (B1*); arrays 13-16 in yellow (B2); arrays 17-20 in blue (B3). Arrays 7-12 were hybridized with an RNA sample derived from batch B1 (as are arrays 1,2,5,6) but amplified separately (hence, denoted B1*).

Analysis: Resulting TIFF images were analyzed with IPLab software (Fairfax, Va.). Cy5/Cy3 ratios of experimental sample intensities to reference intensities for all targets were computed, and ratio normalization performed to set the center of the ratio distribution to 1.0 (13). To assess reliability of each ratio measurement, a quality score ranging from 0 (low) to 1 (high) was determined for each spot location (termed rQuality). PlasmoDB, the Plasmodium Genome Resource Version 5.4 (plasmodb.org/plasmo), was used to acquire annotation information on the sequences of the oligos identified by this analysis.

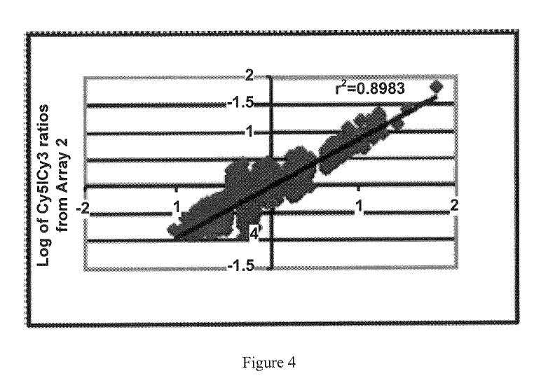

Selection of Arrays: Twenty hybridized and scanned arrays (shown in FIG. 3) were selected for further analysis by the following filtration method. Briefly, a quality score ranging from 0 (low) to 1 (high) was determined for each spot location (termed rQuality) and only data from those spots with rQuality>0.5 were taken into account. Normalized Cy5/Cy3 ratios from each technical replicate were compared within an experimental batch and only arrays with r²>0.80 (when compared to all other technical replicate within the same batch) were selected (FIG. 4 and Table 1). Using these criteria, 8 arrays were chosen for further analysis and these are shown in Table 2 (array 1, 2, 9, 10, 11,17,18, and 19). All arrays from Batch 2 failed our selection criteria and thus are not shown in the figures.

TABLE 1
Selected Technical Replicate Arrays

| | | No. | | | |
|---|---|---|---|---|---|
| Arrays | | spots | | | |
| com- | | with | | | |
| pared¹ | RNA batch | rQ > 0.5 | r² | | |
| 1 vs. 2 | B1 | 1682 | 0.8983 | } | Exp. Batch |
| | | | | | B1 |
| 9 vs. 10 | B1*-aRNA | 1096 | 0.8509 | | |
| | prep different | | | | |
| 9 vs. 11 | B1*-aRNA | 994 | 0.8766 | | Exp. Batch |
| | prep different | | | {close oversize brace} | B1* |
| 10 vs. 11 | B1*-aRNA | 1076 | 0.8417 | | |
| | prep different | | | | |
| 17 vs. 18 | B3 | 1858 | 0.9412 | | |
| 17 vs. 19 | B3 | 768 | 0.8237 | {close oversize brace} | Exp. Batch |
| 18 vs. 19 | B3 | 745 | 0.8516 | | B3 |

¹Arrays 1 and 2 are technical replicates from experimental batch, B1. Arrays 9-11 are technical replicates from experimental batch, B1*. Arrays 17-19 are technical replicates from experimental batch, B3.

TABLE 2
Number of differentially expressed genes in the selected eight
arrays for which all technical replicates had r² > 0.80.

| | | | | No. | No. down- |
|---|---|---|---|---|---|
| | | | | upregulated | regulated |
| | | Array¹ | RNA batch | genes | genes |
| Exp. Batch | | 1 | B1 | 111 | 243 |
| | {close oversize brace} | | | | |
| B1 | | 2 | B1 | 111 | 222 |
| | | 9 | B1*-aRNA | 128 | 140 |
| | | | prep different | | |
| Exp. Batch | | 10 | B1*-aRNA | 131 | 111 |
| B1* | {close oversize brace} | | prep different | | |
| | | 11 | B1*-aRNA | 137 | 108 |
| | | | prep different | | |
| | | 17 | B3 | 154 | 267 |
| Exp. Batch | {close oversize brace} | 18 | B3 | 124 | 226 |
| B3 | | 19 | B3 | 78 | 113 |

¹Arrays 1 and 2 are technical replicates from experimental batch, B1. Arrays 9-11 are technical replicates from experimental batch, B1*. Arrays 17-19 are technical replicates from experimental batch, B3.

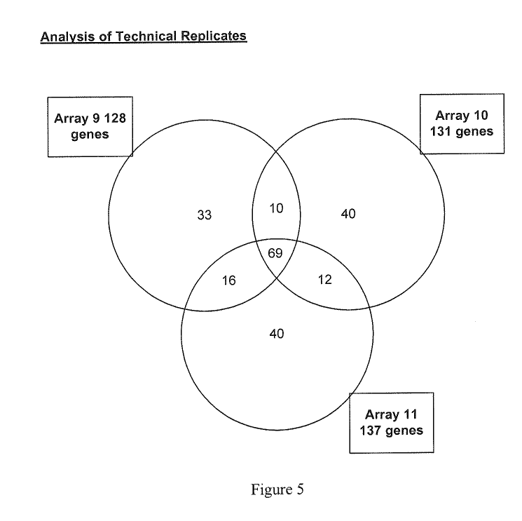

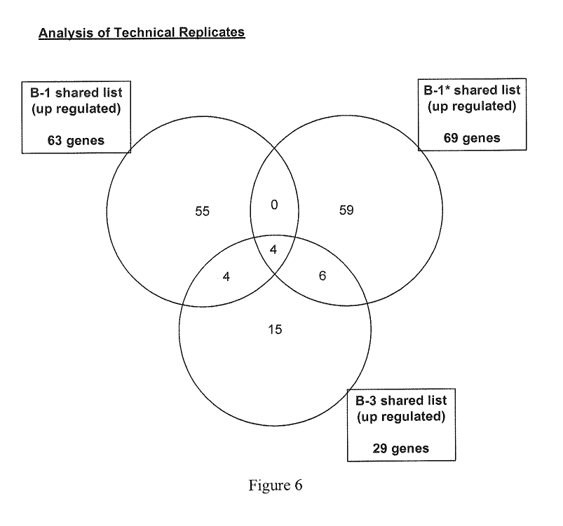

Identification of Differentially Expressed Genes: Differentially regulated loci from each of these 8 gene arrays were chosen on the basis of fold change (i.e. normalized Cy5/Cy3 ratios). The numbers of loci exhibiting fold changes >1.5 in either direction (up or down regulation) in response to radiation is tabulated as shown in Table 2 (above) for each of the 8 arrays. The majority of fold changes observed was low in all samples, ranging from 1.5 to 2.5-fold, with the highest fold changes at only 3-4 fold and only in a few transcripts. Over 50% of the responsive genes were shared among technical replicates within an experimental batch. This is shown for Batch B1* in FIG. 5. However, from the 3 experimental batches tested, only 4 genes reproducibly responded to radiation exposure in the 8 arrays that meet these stringent selection criteria (FIG. 6 and Table 3). All four are up-regulated. Three are genes from well-known functional groups, redox metabolism (redox-PFC0166w), membrane proteins (membrane-PFI1820w) and lipid biosynthesis, (lipid metabolism-MAL8P1.37 proteins). The fourth is a conserved hypothetical protein (PFD0235c).

TABLE 3
Characteristics of the 4 genes which were up-regulated in irradiated P. falciparam sporozoites
as compared to non-irradiated P. falciparum sporozoites in all 8 arrays that met the stringent selection criteria.

| | | | | | Size of | | | | |
|---|---|---|---|---|---|---|---|---|---|
| | | | | | predicted | | | Protein | |
| | | | | Size of | ORF | Chromo- | Microarray | Expression | |
| | | | | predicted | (Amino | some | Expression | Profile | |
| Oligo ID | Gene | Name | Family | RNA (bp) | acids) | Location | Profile | Mass Spec. | Comments |
| C115 | PFC0166w | Putative | Thioredoxin | 540 | 179 | 3 | Erythrocytic | Not available | Redox |
| | | Plasmo- | Superfamily | | | | stage (maximal | | activity shown |
| | | redoxin | | | | | expression at | | |
| | | (Plrx) | | | | | late ring/early | | |
| | | | | | | | trophozoite | | |
| | | | | | | | stage) | | |
| | | | | | | | Expression | | |
| | | | | | | | at schizont | | |
| | | | | | | | stage not | | |
| | | | | | | | assayed | | |
| I14393_1 | PFI1820w | PfEMP1 | Var family | 3948 | 1315 | 9 | Erythrocytic | Not available | Role in |
| | | | of adhesion | | | | stage | | immune |
| | | | proteins | | | | Sporozoite | | evasion |
| | | | | | | | stage | | |
| oPFH0018 | MAL8P1.37 | Putative | Lipoate- | 1242 | 413 | 8 | Erythrocytic | Not available | Post- |
| | | lipoate- | protein | | | | stage (maximal | | translational |
| | | protein | ligase | | | | expression in | | lipoylation |
| | | ligase | | | | | late ring stage) | | |
| F44947_3 | PFD0235c | Hypo- | Not | 1704 | 567 | 4 | Erythrocytic | Gametocyte | None |
| | | thetical | available | | | | stage (maximal | Sporozoite | currently |
| | | protein, | | | | | expression at | | |
| | | conserved | | | | | ring/schizont/ | | |
| | | | | | | | merozoite | | |
| | | | | | | | stage) | | |
| | | | | | | | Sporozoite | | |
| | | | | | | | stage | | |
| | | | | | | | Gametocyte | | |
| | | | | | | | stage | | |

These four sequences are newly identified in playing a role in the protection conferred by radiation attenuated PfSPZ. They are derived from 3 known genes from separate functional groups, including lipid biosynthesis, redox metabolism, and membrane proteins. The fourth sequence is derived from a gene encoding a predicted hypothetical protein.

The four novel genes identified are:

I) PFC0166w, a redox-active protein in P. falciparum, named plasmoredoxin (Plrx), which is highly conserved but found exclusively in malarial parasites. The gene does not have introns, therefore both the DNA and RNA sequences are identified herein as SEQ ID NO:1 and the encoded polypeptide sequence is identified herein as SEQ ID NO:2.

II) PFI1820w a member of the variant (var) gene family that plays a role in adhesion of infected erythrocytes to endothelial cells in the brain and other organs, and is involved in immune evasion. The gene does not have introns, therefore both the DNA and RNA sequences are identified herein as SEQ ID NO:3 and the encoded polypeptide sequence is identified herein as SEQ ID NO:4.

III) MAL8P1.37 a putative lipoate-protein ligase in irradiated sporozoites, which may play an important role in the metabolism and survival of these attenuated forms in hepatocytes. The gene does not have introns, therefore both the DNA and RNA sequences are identified herein as SEQ ID NO:5 and the encoded polypeptide sequence is identified herein as SEQ ID NO:6.

IV) PFD0235c, a conserved hypothetical protein. The gene does not have introns, therefore both the DNA and RNA sequences are identified herein as SEQ ID NO:7 and the encoded polypeptide sequence is identified herein as SEQ ID NO:8.

This limited response (4 genes reproducibly affected) to radiation at the transcript level in sporozoites, is not unexpected, given that irradiated sporozoites do not differ morphologically or physiologically from non-irradiated ones. Both forms can invade hepatocytes, and express liver stage proteins that are not expressed in sporozoites. However, subsequent development is halted in the irradiated sporozoites.

### Example 2

### Preparing a Vaccine

The approach to be taken in preparing a vaccine is straightforward and is known in the art. For example, see references 13-18, incorporated herein by reference.

The steps can include one or more of the following:
1. Clone the gene of interest, as identified in Example 1 above, using standard methods well-known in the art.
2. Produce an immunogen, either as a recombinant protein (13) or as a DNA plasmid (16), or recombinant virus (15, 17, 18) encoding the gene expressing the protein using methods well-known in the art and either described or referenced in the papers referenced.
3. Immunize mice with this immunogen using standard methods, well known to those in the art and described in several of the references (13,14)
4. Establish that antibodies in sera from the immunized mice recognize P. falciparum sporozoites, have biological activity against sporozoites, and/or that the immunization regimen induces T cell responses against the particular protein using standard, established methods, well-known to those in the art and described in the referenced publications and in references in those publications (13-17)
5. Manufacture the immunogen in compliance with FDA regulations.
6. Immunize humans in a clinical trial and determine if the candidate vaccine is safe, immunogenic, and protects against experimental challenge using standard, established methods, well-known to those in the art (5,15).
7. Proceed with development.

In the foregoing, the present invention has been described with reference to suitable embodiments, but these embodiments are only for purposes of understanding the invention and various alterations or modifications are possible so long as the present invention does not deviate from the claims.

### Incorporation by Reference

All of the U.S. patents, U.S. published patent applications, and references cited herein are hereby incorporated by reference.

### Equivalents

Those skilled in the art will recognize, or be able to ascertain using no more than routine experimentation, many equivalents to the specific embodiments of the invention described herein.

### REFERENCES

1. Breman J G, Alilio M S, Mills A. Conquering the intolerable burden of malaria; what's new, what's needed: a summary. Am J Trop Med Hyg. 2004; 71(2 Suppl): 1-15. Review. 15331814
2. Weiss W R, Sedegah M, Beaudoin R L, Miller L H, Good M F. CD8+ T cells (cytotoxic/suppressors) are required for protection in mice immunized with malaria sporozoites. Proc Natl Acad Sci U S A. 1988 January; 85(2):573-6.
3. Hoffman S. L. et al., J. Infect. Dis. (2002) 185: 1155-64
4. Gruner A C, Mauduit M, Tewari R, Romero J F, Depinay N, Kayibanda M, Lallemand E, Chavatte J M, Crisanti A, Sinnis P, Mazier D, Corradin G, Snounou G, Rénia L. Sterile protection against malaria is independent of immune responses to the circumsporozoite protein. PLoS ONE. 2007 Dec. 26; 2(12):e1371.
5. Kester K E, McKinney D A, Tomieporth N, Ockenhouse C F, Heppner D G, Hall T, Krzych U, Delchambre M, Voss G, Dowler M G, Palensky J, Wittes J, Cohen J, Ballou W R; RTS,S Malaria Vaccine Evaluation Group. Efficacy of recombinant circumsporozoite protein vaccine regimens against experimental Plasmodium falciparum malaria. J Infect Dis. 2001 Feb. 15; 183(4):640-7. Epub 2001 Jan. 24.
6. Kumar K A, Sano G, Boscardin S, Nussenzweig R S, Nussenzweig M C, Zavala F, Nussenzweig V. The circumsporozoite protein is an immunodominant protective antigen in irradiated sporozoites. Nature. 2006 Dec. 14; 444(7121):937-40. Epub 2006 December 6
7. Mikolajczak S A, Aly A S, Kappe. Preerythrocytic malaria vaccine development Curr Opin. Infect Dis. 2007; 20(5): 461-6.
8. Hoffman B U and Chattopadhyay R C. Plasmodium falciparum; Effect of radiation on levels of gene transcripts in sporozoites. Expt Parasitol. 2008; 118: 247-252.
9. Hoffman B U and Gunasekera A. Radiation-induced alterations in gene expression of Plasmodium falciparum sporozoites. Late Breakers in Molecular Biology. Annual Meeting, American Society of Tropical Medicine and Hygiene, Philadelphia, Pa. 2007.
10. Bozdech Z, Zhu J, Joachimiak M P, Cohen F E, Pulliam B, DeRisi J L. Expression profiling of the schizont and trophozoite stages of Plasmodium falciparum with a long-oligonucleotide microarray. Genome Biol. 2003:4(2):R9. Epub 2003 Jan. 31. PMID: 12620119
11. Gardner M J, et al. Genome sequence of the human malaria parasite Plasmodium falciparum. Nature, 2002; 419: 498-511.
12. Xiang C C, Kozhich O A, Chen M, Inman J M, Phan Q N and Brownstein M J An improved method to label probes for DNA microarray work: amine-modified random primers. Nat. Biotechnol. 2002; 20:738-742. Chen Y, Kamat V, Dougherty E R, Bittner M L, Meltzer P S and Trent J M. Ration statistics of gene expression levels and applications to microarray data analysis. Bioinformatics. 2002; 18: 1207-1215.
13. Rogers, W. O., Rogers, M. D., Hedstrom, R. C., and Hoffman, S. L. Characterization of the gene encoding sporozoite surface protein 2, a protective Plasmodium yoelli sporozoite antigen. Mol. Biochem. Parasitol. 53:45-51, 1992.
14. Chattopadhyay, R, et al. PfSPATR, a Plasmodium falciparum protein containing an altered Thrombospondin type I repeat domain is expressed at several stages of the parasite life cycle and is the target of inhibitory antibodies. J. Biol. Chem 2003 278:25977-25981.
15. Ockenhouse, C F, et al. Phase I/IIa safety, immunogenicity and efficacy trial of NYVAC-Pf7, a pox-vectored, multiantigen, multistage vaccine candidate for Plasmodium falciparum malaria. J. Infec. Dis. 1998 177:1664-1673.
16. Wang, R., et al. Boosting DNA vaccine-elicited gamma interferon responses in humans by exposure to malaria parasites. Infect and Immun. 2005 73:2863-2872.
17. Prieur, E, et al. A Plasmodium falciparum candidate vaccine based on a six-antigen polyprotein encoded by recombinant poxviruses. PNAS 2004 101:290-295.
18. Aguiar, J. C. et al. High throughput generation of P. falciparum functional molecules by recombinational cloning. Genome. Genome Res. 2009 14:2076-2082.

1. A vaccine for the prevention of malaria, said vaccine comprising a polypeptide immunogen the sequence of which is encoded by a transcript which is up-regulated in radiation attenuated P. falciparum sporozoites, said sequence corresponding to SEQ ID NO:4, SEQ ID NO:6, or SEQ ID NO:8.

2. The vaccine of claim 1 comprising at least 25 micrograms but not more than 75 micrograms of immunogen.

3. The vaccine of claim 1 additionally comprising an adjuvant.

4. The vaccine of claim 2 in which the adjuvant is a mineral salt, an oil emulsion, a surfactant based formulation, a particulate, a microbial derivative, an endogenous human immunomodulator, or an inert vehicle.

5. (canceled)

6. The vaccine of claim 1, said sequence corresponding to SEQ ID NO:4.

7. The vaccine of claim 1, said sequence corresponding to SEQ ID NO:6.

8. The vaccine of claim 1, said sequence corresponding to SEQ ID NO:8.

9. The vaccine of claim 1, comprising at least two polypeptide sequences, each encoded by a transcript which is up-regulated in radiation attenuated P. falciparum sporozoites wherein each sequence is chosen from the group consisting of SEQ ID NO:4, SEQ ID NO:6 and SEQ ID NO:8.

10. A method for providing protective immunity against malaria, said method comprising administration of a vaccine to a subject in need thereof comprising a polypeptide immunogen the sequence of which is encoded by a transcript which is up-regulated in radiation attenuated P. falciparum sporozoites, said sequence corresponding to SEQ ID NO:2, SEQ ID NO:4, SEQ ID NO:6, or SEQ ID NO:8.

11. The method of claim 10 additionally comprising a multi-dose regimen.

12. The method of claim 11 comprising a regimen of 3 doses.

13. The method of claim 10 wherein said vaccine comprises at least 25 micrograms but not more than 75 micrograms of immunogen.

14. The method of claim 10 wherein said vaccine additionally comprises an adjuvant.

15. (canceled)

16. The method of claim 10, said sequence corresponding to SEQ ID NO:4.

17. The method of claim 10, said sequence corresponding to SEQ ID NO:6.

18. The method of claim 10, said sequence corresponding to SEQ ID NO:8.

19. The method of claim 10, said vaccine comprising at least two polypeptide sequences, each encoded by a transcript which is up-regulated in radiation attenuated P. falciparum sporozoites wherein each sequence is chosen from the group consisting of SEQ ID NO:4, SEQ ID NO:6 and SEQ ID NO:8.
